(19) United States
(12) Patent Application Publication
(10) Pub. No.: US 2024/0009262 A1
(43) Pub. Date: Jan. 11, 2024

(54) CLEANSING PAD AND COMPOSITION FOR REDUCING TOXINS

(71) Applicant: UAB Orbio World, Kaunas (LT)

(72) Inventor: Vitalija Kreiviene, Kaunas (LT)

(73) Assignee: UAB Orbio World, Kaunas (LT)

(21) Appl. No.: 17/861,210

(22) Filed: Jul. 9, 2022

(51) Int. Cl. A61K 36/9068; A61K 36/899; A61K 35/66; A61K 33/32; A61K 31/722; A61K 36/73; A61K 31/718; A61K 31/375; A61K 36/78; A61K 9/70; A61K 36/00; A61K 47/10

(52) U.S. Cl. CPC A61K 36/9068; A61K 36/899; A61K 35/66; A61K 33/32; A61K 31/722; A61K 36/73; A61K 31/718; A61K 31/375; A61K 36/78; A61K 9/7084; A61K 36/00; A61K 47/10

(57) ABSTRACT

A composition of matter and a patch infused with the patch arranged to assist in reducing toxins of a body. The composition includes ginger and one or more of bamboo vinegar, wood vinegar, tourmaline, chitin, loquat leaf, dextrin, vitamin C, and houttuynia cordata thumb. The composition may also include amber dust. The patch is arranged for retention of sweat from the body in a patch body thereof.

### BACKGROUND OF THE INVENTION

### 1. Field of the Invention

The present invention relates to a composition and a cleansing pad that can be removably applied to the sole of the foot or elsewhere on the body. More particularly, the present invention relates to a cleansing pad including compositions of matter selected to provide deep body cleansing organically. The composition of the cleansing pad includes ginger, amber dust, and other components.

### 2. Description of the Prior Art

Humans have been suffering with an array of well-being ailments for as long as can be remembered. In the modern era, these ailments are physical and mental. Just a few examples are brain fog, irritability, exhaustion, stress-induced muscle aches, an inability to concentrate, restlessness, and tiredness. Attempts to alleviate these ailments in recent times resulted in the proliferation of synthetic chemical products in the form of pharmaceuticals. These products are ingested internally, have many adverse side effects, and insert foreign substances into the body, which substances may target much more than the ailment for which they were originally consumed. It is desirable to address human ailments while minimizing the use of pharmaceuticals.

Traditional oriental medicines have been around for thousands of years. They include, but are not limited to, natural herbs and combinations of herbs, as well as physical manipulation such as massage and acupuncture. These methods have been widely used with success and avoid the introduction of foreign chemical products and the harm they can generate. The introduction of natural herbs that are ingested or temporarily placed on the body can be effective, but the time of application is fairly limited, and the resultant reduction of a well-being ailment may be temporary. In addition, it can be difficult to characterize the best herb or combination of herbs to achieve the desired outcome.

Acupuncture, for example, is a traditional Japanese method of treatment. It has been determined that roughly 360 acupuncture points exist on the human body. Sixty of them are on the sole of the foot. Unfortunately, acupuncture can be something of a temporary fix as the point activation is effective while the needle is inserted and for a modest time thereafter.

What is needed is a natural approach to treatment of well-being ailments that avoids the adverse impact of synthetic chemicals. Also, what is needed is a composition of natural materials that when combined provide an effective wellness solution. Further, what is needed is a combination of a composition of natural materials and a mechanism for delivering that composition to a human being in a natural way, wherein the mechanism aids in maintaining the composition in contact with the body for a selectable period of time that overcomes the limitations of acupuncture needle application.

### SUMMARY OF THE INVENTION

It is an object of the present invention to provide a natural approach to treatment of well-being ailments that avoids the adverse impact of synthetic chemicals. It is also an object of the present invention to provide a composition of natural materials that when combined provide an effective wellness solution. Another object of the invention is to provide a combination of a composition of natural materials and a mechanism for delivering that composition to a human being in a natural way, wherein the mechanism aids in maintaining the composition in contact with the body for a selectable period of time that overcomes the limitations of acupuncture needle application.

These and other objects are achieved with the present invention. The present includes a composition of natural materials and a mechanism for delivery of that composition to the human body. The composition includes a combination of two or more of loquat leaf, bamboo vinegar, vitamin C, wood vinegar, tourmaline, houttuynia cordata thumb, dextrin, ginger, and amber dust. The mechanism for delivery of the composition is a patch include a body into which the composition is infused, and an adhering surface arranged to permit a user to temporarily apply the body to the skin and substantially remain in place until the user intentionally removes it.

The natural materials of the composition are selected to induce sweating at the location of application of the patch. The natural materials provide a pathway for toxins in the body to pass with the sweat onto the patch. As a sauna can be useful in generating sweat that may aid in evacuating toxins from the body, the combination of patch and composition of the present invention facilitate the removal of toxins through the sweat glands but without the need to invest in a sauna or travel to a sauna. In addition, the natural materials selected to be the ingredients of the composition provide an improved sweat generation and toxin removal. Some of the ingredients, such vitamins but not limited thereto, are also infused into the body, and thereby strengthen and/or supplement the body's natural toxin-defeating agents.

The present invention comprising the natural ingredients composition and the composition-delivering patch improve well-being characteristics of the user. The ingredients are natural and proven to be of little to no hazard for the user. The invention is more fully described with respect to the following detailed description, accompanying drawings, and appended claims.

### BRIEF DESCRIPTION OF THE DRAWINGS

FIG. 1 is a top perspective view of the ingredient delivering patch of the present invention.

FIG. 2 is a cross-sectional side view of the patch of FIG. 1, showing a contact-adhesion layer with removable cover, a patch body, and a patch body sealing layer.

FIG. 3 is a perspective view showing a first step of application of the patch to the bottom of a foot.

FIG. 4 is a perspective view showing the patch applied to the bottom of the foot.

FIG. 5 is a plan view showing toxin removal outcome over a series of three applications to the bottom of the foot.

### DETAILED DESCRIPTION OF THE INVENTION

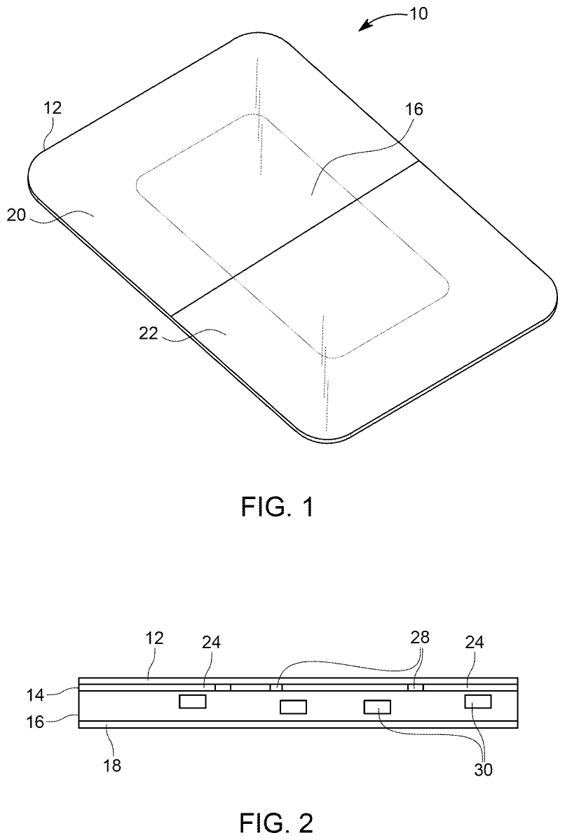

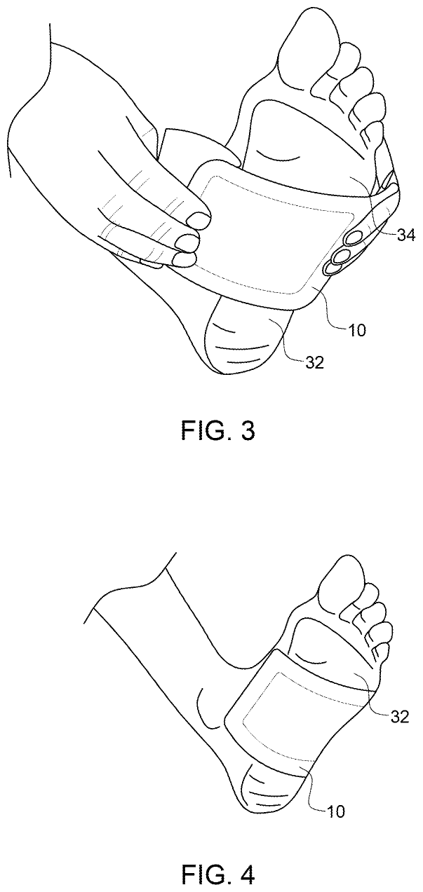

A natural materials delivery mechanism in the form of a patch 10 is shown in FIGS. 1 and 2. The patch 10 includes a removable cover 12, a patch adhesion layer 14, a patch body 16, and a patch body sealing layer 18. The removable cover 12 includes a first section 20 and a second section 22. The first section 20 and the second section 22 are made of non-adhering material, such as silicone paper, non-woven fabric, a combination thereof, or other materials known to those of skill in the art. The non-adhering material is a nonmetallic material having flexibility. The removable cover 12 is a release paper design to cover the patch adhesion layer 14 so that the patch 10 may be positioned spaced from a contact location of interest, such as the bottom of the foot but not limited thereto without unintended adherence to the location. The removable cover 12 may be peeled from surface 24 of the patch adhesion layer 14.

The patch adhesion layer 14 is a thin layer of adhesive material that is preferably hypoallergenic but does not have to be. The patch adhesion layer 14 is made of a hot-melt adhesive but not limited thereto. It is selected to ensure that the patch 10 remains in place on the location of interest while also being removable when toxin-removal activity is completed. The removable cover 12 and the patch adhesion layer 14 perform much like the release paper and adhesive surface of a bandage. The patch adhesion layer 14 includes adhesive material 26 as well as micropores 28 that enable the passage of sweat and any toxins therein through the patch adhesion layer 14 to the patch body 16, and the passage of one or more ingredients of a composition infused to the patch body to pass through pores of the skin.

The patch body 16 is formed of a non-metallic material having features that enable the retention therein of sweat with any toxins. Those features also enable the release of at least a portion of the composition through the pores of the skin. The features of the patch body 16 that enable such material exchange include cells 30 of the patch body 16. The patch body 16 may be formed of a foam material that is an open-cell foam with cell 30 size sufficient to retain at least a portion of the composition infused therein and to recover sweat drops therein. The patch body may be made of a non-woven fabric. The patch body 16 may also be configured as a pocket with sealed or joined edges, with the composition contained therein.

The patch body sealing layer 18 may cover a portion or the entirety of the patch body 16. It may be integrated with the patch adhesion layer 14. It is arranged to maintain the patch 10 in a stored manner while retaining the natural materials of the composition therein until such time as the patch 10 is applied to the skin. The patch body sealing layer 18 is fabricated of a non-metallic material such as a non-woven fabric but not limited thereto. The patch body sealing layer 18 is made of a flexible material so that the patch 10 may be formed to a location of interest, which may itself be flexible as well as a non-flat surface.

The patch body 16 is infused with two or more natural ingredients selected to facilitate sweat withdrawal from the skin and to supply the body with beneficial ingredients such as vitamins but not limited thereto.

In a first embodiment of the composition of the invention, the composition of natural ingredients includes ginger and one or more of bamboo vinegar, wood vinegar, tourmaline, chitin, loquat leaf, dextrin, vitamin C, and houttuynia cordata thumb. The ginger may be about 4% to about 8% by weight of the composition, the bamboo vinegar may be about 25% to about 45% by weight of the composition, the wood vinegar may be about 20% to about 40% by weight of the composition, the tourmaline may be about 0% to about 4% by weight of the composition, the chitin may be about 0% to about 10% by weight of the composition, the loquat leaf may be about 0% to about 15% by weight of the composition, the dextrin may be about 0% to about 10% by weight of the composition, the vitamin C may be about 0% to about 20% by weight of the composition, and the houttuynia cordata thumb may be about 0% to about 20% by weight of the composition. Additionally, an ingredients combining material may be used to make up the weight difference of the composition and to act as a way to keep the ingredients joined together. That combining material may be polyethylene glycol in solid form, such as a powder, but not limited thereto. The combining material may be about 0% to about 10% by weight of the composition.

In a specific example of the first embodiment of the composition, The ginger is about 6% by weight of the composition, the bamboo vinegar is about 35% by weight of the composition, the wood vinegar is about 30% by weight of the composition, the tourmaline is about 2% by weight of the composition, the chitin is about 5% by weight of the composition, the loquat leaf is about 7% by weight of the composition, the dextrin is about 5% by weight of the composition, the vitamin C is about 8% by weight of the composition, and the houttuynia cordata thumb is about 8% by weight of the composition. The first embodiment of the composition may also include amber dust in a range of about 15% to about 25% by weight of the composition. Additionally, the ingredients combining material may be used to make up the weight difference of the composition and to act as a way to keep the ingredients joined together. That combining material may be polyethylene glycol that forms about 4% by weight of the composition.

In a second embodiment of the composition of the invention, the composition of natural ingredients includes amber dust and one or more of bamboo vinegar, wood vinegar, tourmaline, chitin, loquat leaf, dextrin, vitamin C, and houttuynia cordata thumb. The amber dust may be about 15% to about 25% by weight of the composition, the bamboo vinegar may be about 25% to about 45% by weight of the composition, the wood vinegar may be about 20% to about 40% by weight of the composition, the tourmaline may be about 0% to about 4% by weight of the composition, the chitin may be about 0% to about 10% by weight of the composition, the loquat leaf may be about 0% to about 15% by weight of the composition, the dextrin may be about 0% to about 10% by weight of the composition, the vitamin C may be about 0% to about 20% by weight of the composition, and the houttuynia cordata thumb may be about 0% to about 20% by weight of the composition. Additionally, the ingredients combining material, such as polyethylene glycol, may be used to make up the weight difference of the composition and to act as a way to keep the ingredients joined together. The combining material may be about 0% to about 10% by weight of the composition.

In a specific example of the second embodiment of the composition, The amber dust is about 20% by weight of the composition, the bamboo vinegar is about 35% by weight of the composition, the wood vinegar is about 30% by weight of the composition, the tourmaline is about 2% by weight of the composition, the chitin is about 5% by weight of the composition, the loquat leaf is about 7% by weight of the composition, the dextrin is about 5% by weight of the composition, the vitamin C is about 8% by weight of the composition, and the houttuynia cordata thumb is about 8% by weight of the composition. The second embodiment of the composition may also include ginger in a range of about 4% to about 8% by weight of the composition. Additionally, the ingredients combining material, such as polyethylene glycol, optionally forms about 4% by weight of the composition.

The selected composition is formed by mixing the ingredients in dry form in a container suitable for the quantity to be used to infuse or insert into as many patches 10 as desired. For example, the container may be a bowl or jar. The mixing may be accomplished manually or in an automated environment, such as in a mixing and filling machine of the type used to mix solids in powder or other form together. The mixed composition is then inserted into the patch 10 and the patch 10 further processed as needed to retain the composition therein while enabling that composition to interact with the skin.

The patch 10 of the present invention may be applied to a location of interest, such as a body part, such as a sole of a foot, but not limited thereto. The sole of the foot may be advantageous given its nerve sensitivity as evidenced by acupuncture procedures, but not limited thereto. FIGS. 3 and 4 depict primary steps of placing the patch 10 in a location of interest. In this example, the location of interest is a sole 32 of a foot 34. The patch 10 may be placed at other locations on the skin and a plurality of patches may be used as the same time in two or more distinct locations, such as the soles of both feet but not limited thereto.

In use, the first section 20 and the second section 22 of the removable cover 12 are removed from the patch adhesion layer 14. The patch adhesion layer 14 is then placed on the sole 32 of the foot 34. The patch 10 is then smoothed out so that the patch body 16 is substantially in contact with the sole 32. The patch 10 is then left on the location of interest for a selectable period of time, such as six to eight hours but not limited thereto. The patch 10 is then removed when the time period selected for patch attachment to the sole 32 has been completed.

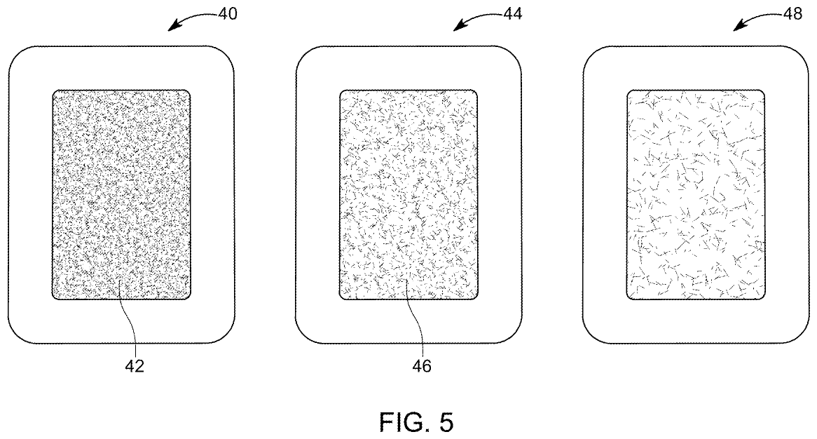

FIG. 5 depicts a progression of toxin removal from the body from a first day to a tenth day of patch application to a location of interest, such as the sole of the foot. The composition infused to the patch body 16 of FIG. 5 is formed of amber dust about 20% by weight of the composition, bamboo vinegar about 35% by weight of the composition, wood vinegar is about 30% by weight of the composition, tourmaline is about 2% by weight of the composition, chitin about 5% by weight of the composition, loquat leaf about 7% by weight of the composition, dextrin about 5% by weight of the composition, vitamin C about 8% by weight of the composition, houttuynia cordata thumb about 8% by weight of the composition, and ginger about 6% by weight of the composition.

It can be seen in a first stage 40 that is a first day of patch 10 application for about 6-8 hours, that a substantial amount of toxin in the form of dark material 42 has been withdrawn from the body. In a second stage 44 that is a fifth day of patch 10 application for about 6-8 hours, that a lesser amount of toxin material in the form of beige material 46 has been withdrawn from the body. Finally, in a third stage 48 that is a tenth day of patch 10 application for about 6-8 hours, that very little toxin material has been withdrawn as there is likely very little sweat inducement occurring by that date.

The invention has been described with specific examples and embodiments. It is not limited thereto but is instead defined by the appended claims and reasonable equivalents.

What is claimed is:

1. A composition of matter made to assist in reducing toxins of a body, the composition comprising:
ginger and one or more of bamboo vinegar, wood vinegar, tourmaline, chitin, loquat leaf, dextrin, vitamin C, and houttuynia cordata thumb.

2. The composition of claim 1, wherein the ginger is about 4% to about 8% by weight of the composition, the bamboo vinegar is about 25% to about 45% by weight of the composition, the wood vinegar is about 20% to about 40% by weight of the composition, the tourmaline is about 0% to about 4% by weight of the composition, the chitin is about 0% to about 10% by weight of the composition, the loquat leaf is about 0% to about 15% by weight of the composition, the dextrin is about 0% to about 10% by weight of the composition, the vitamin C is about 0% to about 20% by weight of the composition, and the houttuynia cordata thumb is about 0% to about 20% by weight of the composition.

3. The composition of claim 2, wherein the ginger is about 6% by weight of the composition, the bamboo vinegar is about 35% by weight of the composition, the wood vinegar is about 30% by weight of the composition, the tourmaline is about 2% by weight of the composition, the chitin is about 5% by weight of the composition, the loquat leaf is about 7% by weight of the composition, the dextrin is about 5% by weight of the composition, the vitamin C is about 8% by weight of the composition, and the houttuynia cordata thumb is about 8% by weight of the composition.

4. The composition of claim 1 further comprising amber dust in a range of about 15% to about 25% by weight of the composition.

5. The composition of claim 1 further comprising an ingredients combining material in a range of about 0% to about 10% by weight of the composition.

6. The composition of claim 5 wherein the ingredients combining material is polyethylene glycol comprising about 4% by weight of the composition.

7. A composition of matter made to assist in reducing toxins of a body, the composition comprising:
amber dust and one or more of bamboo vinegar, wood vinegar, tourmaline, chitin, loquat leaf, dextrin, vitamin C, and houttuynia cordata thumb.

8. The composition of claim 7, wherein the amber dust is about 15% to about 25% by weight of the composition, the bamboo vinegar is about 25% to about 45% by weight of the composition, the wood vinegar is about 20% to about 40% by weight of the composition, the tourmaline is about 0% to about 4% by weight of the composition, the chitin is about 0% to about 10% by weight of the composition, the loquat leaf is about 0% to about 15% by weight of the composition, the dextrin is about 0% to about 10% by weight of the composition, the vitamin C is about 0% to about 20% by weight of the composition, and the houttuynia cordata thumb is about 0% to about 20% by weight of the composition.

9. The composition of claim 8, wherein the amber dust is about 20% by weight of the composition, the bamboo vinegar is about 35% by weight of the composition, the wood vinegar is about 30% by weight of the composition, the tourmaline is about 2% by weight of the composition, the chitin is about 5% by weight of the composition, the loquat leaf is about 7% by weight of the composition, the dextrin is about 5% by weight of the composition, the vitamin C is about 8% by weight of the composition, and the houttuynia cordata thumb is about 8% by weight of the composition.

10. The composition of claim 7 further comprising ginger in a range of about 4% to about 8% by weight of the composition.

11. The composition of claim 7 further comprising an ingredients combining material in a range of about 0% to about 10% by weight of the composition.

12. The composition of claim 11 wherein the ingredients combining material is polyethylene glycol comprising about 4% by weight of the composition.

13. A natural treatment patch for removable application to skin to assist in reducing toxins from the body, the patch comprising:
a patch body; and
an adhesive for removably joining the patch body to the skin, wherein, the patch body is infused with a composition, the composition comprising:
ginger and one or more of bamboo vinegar, wood vinegar, tourmaline, chitin, loquat leaf, dextrin, vitamin C, and houttuynia cordata thumb.

14. The patch of claim 13, wherein the ginger is about 4% to about 8% by weight of the composition, the bamboo vinegar is about 25% to about 45% by weight of the composition, the wood vinegar is about 20% to about 40% by weight of the composition, the tourmaline is about 0% to about 4% by weight of the composition, the chitin is about 0% to about 10% by weight of the composition, the loquat leaf is about 0% to about 15% by weight of the composition, the dextrin is about 0% to about 10% by weight of the composition, the vitamin C is about 0% to about 20% by weight of the composition, and the houttuynia cordata thumb is about 0% to about 20% by weight of the composition.

15. The patch of claim 14, wherein the ginger is about 6% by weight of the composition, the bamboo vinegar is about 35% by weight of the composition, the wood vinegar is about 30% by weight of the composition, the tourmaline is about 2% by weight of the composition, the chitin is about 5% by weight of the composition, the loquat leaf is about 7% by weight of the composition, the dextrin is about 5% by weight of the composition, the vitamin C is about 8% by weight of the composition, and the houttuynia cordata thumb is about 8% by weight of the composition.

16. The patch of claim 13 wherein the composition further includes amber dust in a range of about 15% to about 25% by weight of the composition.

17. The patch of claim 13 wherein the composition further includes an ingredients combining material in a range of about 0% to about 10% by weight of the composition.

18. The composition of claim 17 wherein the ingredients combining material is polyethylene glycol comprising about 4% by weight of the composition.
